(19) United States
(12) Patent Application Publication
(10) Pub. No.: US 2018/0192702 A1
(43) Pub. Date: Jul. 12, 2018

(54) ELECTRONIC CIGARETTE ATOMIZER EQUIPPED WITH A RECORDING CHIP AND AN ELECTRONIC CIGARETTE EQUIPPED WITH SAID ATOMIZER AS WELL AS ITS CONTROL METHOD

(71) Applicant: SHENZHEN INNOKIN TECHNOLOGY CO., LTD, Shenzhen (CN)

(72) Inventor: Jian Wei LI, Shenzhen (CN)

(73) Assignee: SHENZHEN INNOKIN TECHNOLOGY CO. LTD.

(21) Appl. No.: 15/867,021

(22) Filed: Jan. 10, 2018

(30) Foreign Application Priority Data
Jan. 10, 2017 (CN) 201710016022.0
Feb. 22, 2017 (FR) 1751405

(51) Int. Cl. A24F 47/00; A61M 15/06; A61M 15/00

(52) U.S. Cl. CPC A24F 47/008; A61M 15/0065; A61M 15/06

(57) ABSTRACT

An electronic cigarette atomizer is provided. The atomizer may comprise a recording chip. The recording chip may be configured to record identification information of the electronic cigarette atomizer, send said identification information of the electronic cigarette atomizer to an electronic cigarette controller, record operating information of the electronic cigarette atomizer, said operating information comprising service life and number of uses of the core of the atomizer, and send said operating information to the electronic cigarette controller. The identification information of the electronic cigarette atomizer may include identification information of an aromatic liquid and identification information of a core of the atomizer. In another embodiment, an electronic cigarette is provided. In yet another embodiment a method for controlling an atomizer of electronic cigarette is provided.

### CROSS-REFERENCE TO RELATED APPLICATION

This application claims priority to, and incorporates by reference herein in their entireties, Chinese Patent Application No. 201710016022.0, filed on Jan. 10, 2017, and French Patent Application No. 1751405, filed on Feb. 22, 2017.

### TECHNICAL FIELD

The present disclosure relates to the technical field of the manufacture of electronic cigarettes (E-cigarettes, e-Cig, personal vaporizer), and in particular an electronic cigarette atomizer equipped with a recording chip and an electronic cigarette equipped with said atomizer as well as its control method.

### BACKGROUND

The structure of electronic cigarettes generally comprises a device for supplying electrical power to the electronic cigarette (e.g., a power source, such as a battery); an electronic cigarette control circuit (e.g., an electronic cigarette controller such as a microprocessor); an aromatic liquid (e.g., E-liquid, e-juice, smoke juice); a supply device for the electronic cigarette, which may commonly be referred to as a cartridge or tank; a vapor aspiration device; and an electronic cigarette aromatic liquid atomization device (e.g., an electronic cigarette atomizer or heating element). An electronic cigarette atomizer may generally consist of an atomization chamber, and such atomization chamber may be equipped with a core configured to atomize the aromatic liquid of the electronic cigarette by heating it. The core of the atomizer may be equipped with positive and negative poles connected to the control circuit of the electronic cigarette. The core of the atomizer may generally consist of an outer shell and internal heating wires, the internal heating wires being further equipped with an aromatic liquid transport and storage element of the electronic cigarette. Such element may be, for example, formed as a E-liquid guiding body, generally designed in the form of a cotton cord, connected to the aromatic liquid reservoir of the electronic cigarette situated on the outside via a liquid inlet opening arranged on the outer wall of the outer shell of the core of the atomizer. Such element may facilitate a permeation effect of the E-liquid guiding body and carry the aromatic liquid to the heating wires of the core of the atomizer. The heating wires may be equipped with positive and negative poles respectively connected to the positive and negative poles of the electric power supply via conductive wires and conductive columns.

E-liquid guiding bodies that equip current atomizers and those of the last few years are typically formed of cotton or non-woven fabrics; they may be directly in contact with the heating wires. The operating temperature of the heating wires may be between 150° C. and 315° C., and may be even higher if the E-liquid guiding bodies are dry. The resistance of a cotton E-liquid guiding body to high temperatures is low; its charring and inflammation temperatures are very low; and under normal conditions of aromatic liquid supply, the service life of a cotton E-liquid guiding body may be limited. First, when used for a long period, a cotton E-liquid guiding body may lose its elasticity; consequently its transport and aromatic liquid storage functionality may fail, causing leakage in the atomizer as well as leakage phenomena of aromatic liquid in the outlet duct. Second, a cotton E-liquid guiding body may char when it has been used for a long period, causing carbon accumulation at the surface of the heating wires. This may impair the taste provided to the user and, worse, has the potential to produce noxious substances! In this condition, the core of the atomizers can no longer be used, as they depart totally from the purpose of the electronic cigarette.

### SUMMARY

In order to resolve the technical problems described above, the technical effects to be achieved by the present disclosure include supplying an electronic cigarette atomizer equipped with a recording chip; allowing practical identification of identification information of the atomizer, such as information on the aromatic liquid and information on the core of the atomizer; and/or allowing continuous recording of information on the operation of the atomizer such as service life and the number of uses of the core of the atomizer. Such effects advantageous permit improved replacement the atomizer, improved refill of aromatic liquid, improved maintenance of the atomizer in normal operating condition, and/or avoidance of malfunction of undesired operation of the electronic cigarette due to a poor match between an atomizer core and e-liquid or other incorrect use.

In one embodiment, an electronic cigarette atomizer is provided. The atomizer may comprise a recording chip. The recording chip may be configured to record identification information of the electronic cigarette atomizer, send said identification information of the electronic cigarette atomizer to an electronic cigarette controller, record operating information of the electronic cigarette atomizer, said operating information comprising service life and number of uses of the core of the atomizer, and send said operating information to the electronic cigarette controller. The identification information of the electronic cigarette atomizer may include identification information of an aromatic liquid and identification information of a core of the atomizer.

The identification information of the core of the atomizer may include information concerning a material of heating wires of the core of the atomizer, information concerning resistance values of the heating wires of the core of the atomizer; and information concerning aromatic liquids suitable for the core of the atomizer.

The recording chip may include a first information recorder configured to record the identification information of the electronic cigarette atomizer, a second information recorder configured to record the operating information, and an information transmitter configured to send the recorded identification information of the electronic cigarette atomizer and the operating information to the electronic cigarette controller.

The electronic cigarette atomizer may further include an aromatic liquid cartridge. The aromatic liquid cartridge may be equipped with the first information recorder.

The electronic cigarette atomizer may further include an information identifier. An information marker may be provided with an aromatic liquid cartridge. When the aromatic liquid cartridge with the information marker is installed with said electronic cigarette atomizer, the information identifier may be configured to identify said information marker and acquire the identification information of the aromatic liquid in the aromatic liquid cartridge from the information marker.

The information marker may be a barcode, a QR code or a magnetic stripe. The information identifier may be a barcode identifier, a QR code identifier, or a magnetic stripe identifier.

The second information recorder may be configured to be connected to the core of the atomizer.

The identification information of the aromatic liquid may include information on the composition and the quantity of the aromatic liquid.

In another embodiment, an electronic cigarette is provided. The electronic cigarette may include an electronic cigarette atomizer equipped with a recording chip, and an electronic cigarette controller. The recording chip may be configured to record identification information of the electronic cigarette atomizer, send said identification information to the electronic cigarette controller, record operating information of the electronic cigarette atomizer, and to send said operating information on the atomizer to the electronic cigarette controller. The operating information may include service life and number of uses of the core of the atomizer. The identification information may include information on an aromatic liquid and information on a core of the atomizer;

The information on the core of the atomizer may include information concerning the material of heating wires of the core of the atomizer; information concerning resistance values of the heating wires of the core of the atomizer; and information concerning aromatic liquids suitable for the core of the atomizer.

The electronic cigarette controller may be configured to receive the identification information of the electronic cigarette atomizer from the recording chip, identify and authenticate the received identification information, control the operation of the electronic cigarette atomizer if the identification and authentication are successful, prevent the operation of the electronic cigarette atomizer if the identification and authentication fail, receive the operating information of the atomizer from the recording chip, determine if the core of the atomizer is approaching or has exceeded its service life, and stop the operation of the electronic cigarette atomizer if the core of the atomizer has exceeded its service life.

The electronic cigarette controller may further include an identifier-authenticator. The identifier-authenticator may be configured to receive the identification information, compare the identification information with predefined identification information of the electronic cigarette atomizer to assess a proper match between the atomizer core and the aromatic liquid, and if the match is assessed be proper, regulate operating parameters of the core of the atomizer depending on the received identification information to control the operation of the electronic cigarette atomizer. The operating parameters may include an operating power, a current of the core of the atomizer, and a temperature of the core of the atomizer.

The electronic cigarette controller may further include a service life overrunning controller. The service life overrunning controller may be configured to acquire the operating information, compare the operating information with predefined threshold values of duration and/or threshold values of number of uses, and interrupt the operation of the electronic cigarette atomizer if the operating information exceeds the predefined threshold values of duration and/or threshold values of number of uses.

The electronic cigarette may further include a user-perceptible output indicator. The electronic cigarette controller may be further to control the user-perceptible output indicator to provide an indication of the results of the identification and authentication if the identification information of the atomizer is identified and authenticated. The electronic cigarette controller may be further to control the user-perceptible output indicator to provide an indication that the core of the atomizer is approaching or has exceeded its service life if the operating information exceeds the predefined threshold values of duration and/or threshold values of number of uses.

The user-perceptible output indicator may include at least one of a display screen, an indicator light, an auditory output element, and a tactile feedback element.

In yet another embodiment, a method for controlling an atomizer of electronic cigarette is provided. The method may include acquiring and recording identification information of the electronic cigarette atomizer, sending said identification information of the electronic cigarette atomizer to an electronic cigarette controller, acquiring and recording operating information of the electronic cigarette atomizer, and sending said operating information to the electronic cigarette controller. The operating information may include service life and number of uses of the core of the atomizer. The identification information of the electronic cigarette atomizer may include identification information of an aromatic liquid and identification information of a core of the atomizer.

The method may further include receiving the identification information of the electronic cigarette atomizer, identifying and authenticating the received identification information of the electronic cigarette atomizer, controlling the operation of the electronic cigarette atomizer if the identification and authentication are successful, preventing the operation of the electronic cigarette atomizer if the identification and authentication fail, receiving the operating information of the atomizer, determining if the core of the atomizer is approaching or has exceeded its service life, and stopping the operation of the electronic cigarette atomizer if the core of the atomizer has exceeded its service life.

The identification information of the core of the atomizer may include information concerning a material of heating wires of the core of the atomizer, information concerning resistance values of the heating wires of the core of the atomizer, and information concerning aromatic liquids suitable for the core of the atomizer.

The method may further include comparing the identification information of the electronic cigarette atomizer with predefined identification information of the electronic cigarette atomizer to assess a proper match between the atomizer core and the aromatic liquid. If the match is assessed be proper, the method may include regulating operating parameters of the core of the atomizer depending on the received identification information to control the operation of the electronic cigarette atomizer. The operating parameters may include an operating power, a current of the core of the atomizer, and a temperature of the core of the atomizer.

The method may further include comparing the operating information with predefined threshold values of duration and/or threshold values of number of uses, and interrupting the operation of the electronic cigarette atomizer and/or providing and indication to the user if the operating information exceeds the predefined threshold values of duration and/or threshold values of number of uses.

The beneficial effects of the abovementioned technical solutions of the present disclosure include the following: Due to the configuration of the recording chip, it is possible to acquire and to send identification information the operating information of the electronic cigarette atomizer; and to receive and process such information via the electronic cigarette controller. In turn, the disclosure advantageously teaches instrumentalities and methods to identify and authenticate the identification information of the aromatic liquid and the identification information of the core of the atomizer; to check the aging of the core of the atomizer depending on the duration of operation and the number of uses of the core of the atomizer; to prevent the occurrence of leaks of the atomizer; to prevent poor matches between an atomizer core and e-liquid; to prevent aging problems of the core of the atomizer; and to generally improve the safety of the atomizer of the electronic cigarette and user experience.

### BRIEF DESCRIPTION OF THE DRAWINGS

FIG. 1 depicts a schematic diagram of a recording chip of an atomizer and a controller of an electronic cigarette, consistent with exemplary embodiments; and

FIG. 2 shows the steps of a method for controlling an atomizer of an electronic cigarette equipped with a recording chip, consistent with exemplary embodiments.

### DETAILED DESCRIPTION

Below, a detailed description referencing the appended figures and embodiments is provide to clarify the technical problem to be resolved, the technical concept, and the advantages of the present disclosure.

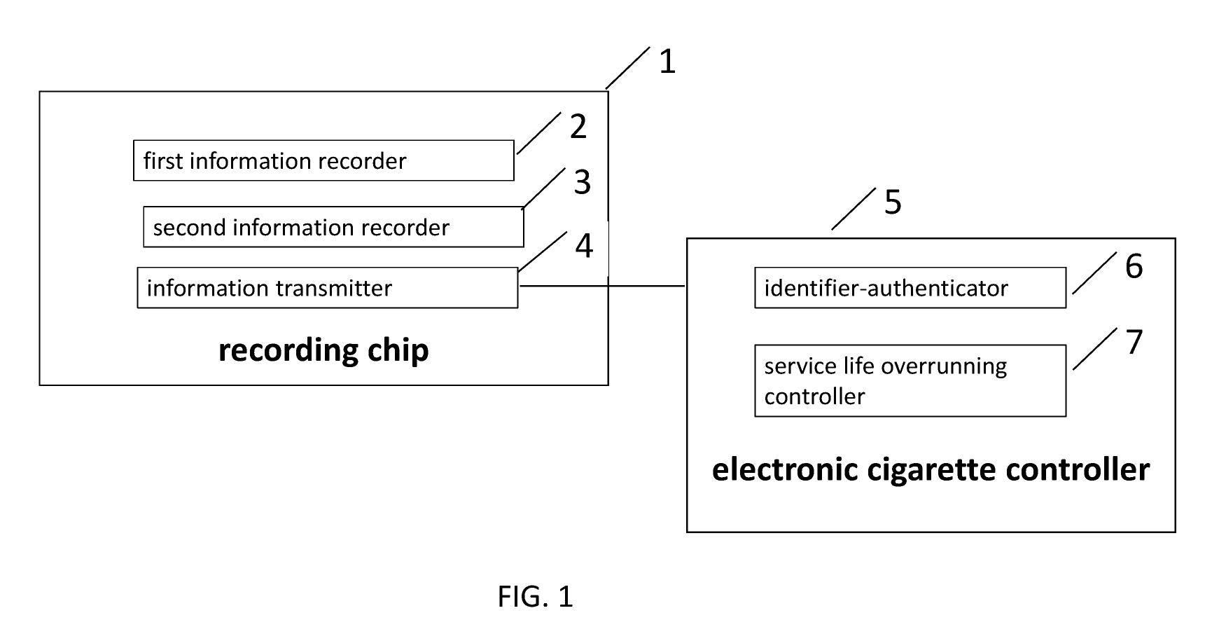

An embodiment of the present disclosure, as shown in FIG. 1, relates to an electronic cigarette atomizer equipped with a recording chip. Said recording chip 1 may be designed to acquire and record the identification information of the electronic cigarette atomizer, said identification information comprising at least identification information on the aromatic liquid and identification information on the core of the atomizer of the electronic cigarette; to send said identification information of the electronic cigarette atomizer to the electronic cigarette controller 5; to record the operating information of the electronic cigarette atomizer, said operating information comprising at least the service life and the number of uses of the core of the atomizer; and to send said operating information of the atomizer to the electronic cigarette controller 5.

The recording chip may include a first information recorder 2 configured to acquire and record the identification information of the atomizer. This identification information may include information of the aromatic liquid and information of the core of the atomizer. Information of the aromatic liquid may include information on the composition of the aromatic liquid and information on the quantity of the aromatic liquid. Information of the core of the atomizer may include information on the material of the heating wires, information on the resistance values of the heating wires, and information concerning the aromatic liquid suitable for the core of the atomizer.

The recording chip may include a second information recorder 3, configured to acquire and record operating information of the atomizer, said information comprising at least the service life and the number of uses of the core of the atomizer.

The recording chip may also include an information transmitter 4, configured to send the acquired identification information of the atomizer and the recorded operating information of the atomizer to the electronic cigarette controller 5.

Physically, recording chip 1 may arranged inside the electronic cigarette atomizer (not shown in the Figures). Recording chip 1 may be connected to the core of the atomizer. Recording chip 1 may be connected to the two poles of the electronic cigarette atomizer so as to supply it with power. Recording chip 1 may be further provided with a third pole arranged on the electronic cigarette atomizer, and may use the connection of the information transmitter 4 with the third pole to be connected to the electronic cigarette controller.

A preferred embodiment supplies a first information recorder 2, a second information recorder 3, and an information transmitter 4. This may allow for rapid and precise acquisition of information on the aromatic liquid and on the core of the atomizer, for example relating to service life and number of uses of the core of the atomizer, and sending such information to the electronic cigarette controller 5. In turn, this may facilitate determining the identification of the aromatic liquid and the core of the atomizer by the electronic cigarette controller, whether the aromatic liquid and the atomizer core are properly matched, and the assessing if there is an overrun of service life of the core of the atomizer.

An electronic cigarette comprising an electronic cigarette atomizer equipped with the abovementioned recording chip 1 is also disclosed. The interior of said electronic cigarette may be equipped with an electronic cigarette controller 5. The electronic cigarette controller 5 may be connected to the electric power supply device of the electronic cigarette—e.g., a power supply source, such as a battery—and control the elements of the electronic cigarette so that it operates properly.

In preferred embodiments, said electronic cigarette controller 5 may also serve to receive the identification information of said atomizer. This identification information may include at least the information of the aromatic liquid and the information of the core of the atomizer. Such identification information may be used to identify and authenticate the identification information of the atomizer; to control the operation of the electronic cigarette atomizer if the identification and authentication are successful; and to stop the operation of the electronic cigarette atomizer if the identification and authentication fail. The electronic cigarette controller 5 may also serve to receive the operating information of said atomizer. This operating information may include at least the service life and the number of uses of the core of the atomizer. Such operating information may be used to determine if the core of the atomizer is approaching or has overrun its service life, and to stop the operation of the electronic cigarette atomizer if the core of the atomizer has overrun its service life.

In some embodiments, the electronic cigarette controller 5 includes an identifier-authenticator 6. Identifier-authenticator 6 may be configured to receive the identification information, to compare identification information with the predefined identification information values to assess an appropriate match between the atomizer core and the aromatic liquid; and to regulate the operating parameters of the core of the atomizer depending on the received identification information of the electronic cigarette atomizer so as to control the operation of the electronic cigarette atomizer if said comparison indicated an appropriate matching. The operating parameters may include at least the operating power, the current, and the temperature of the core of the atomizer.

In preferred embodiments, the setting of the identifier-authenticator 6 enables rapid and practical identification the aromatic liquid and the core of the atomizer, and thus evaluation and the aromatic liquid and the core of the atomizer. In this manner, the electronic cigarette controller 5 may thereby assess if the electric power supply device of the core of the atomizer is suitable for the aromatic liquid, and enable the provision of adequate electric power as appropriate.

Electronic cigarette controller 5 may further include service life overrunning controller 7. Service life overrunning controller 7 may be configured to acquire the operating information of the atomizer, to compare the operating information of the atomizer with the predefined threshold values of service life and the threshold values of number of uses, and to stop the operation of the electronic cigarette atomizer when the operating information of the atomizer exceeds the predefined threshold values of service life and/or threshold values of number of uses. In some embodiments, service life overrunning controller 7 may propose the replacement of the core of the atomizer under certain circumstances.

Service life overrunning controller 7 may be arranged to effectively control the service life and the number of uses of the core of the atomizer, and thereby prevent disturbances linked to the aging of the core of the atomizer.

In some embodiments, said arrangement may include a display configured to display information relating to the atomizer. For example, the display may provide an indication of the results of identification and authentication when the information of the aromatic liquid and the information of the core of the atomizer are identified and authenticated; the operating information of the atomizer when the core of the atomizer approaches or exceeds its service life; and/or if the core of the atomizer must be replaced. These indications may be provided to the user before the operation of the atomizer of the electronic cigarette is interrupted, for example, visually via a screen or an indicator light; audibly via a warning sound; and/or via tactile feedback, such as vibration.

The atomizer of the electronic cigarette may be connected to the electrical power supply, and the electronic cigarette controller may be connected to recording chip 1. Recording chip 1 may record and acquire the identification information of the atomizer, for example, including the identification information of the aromatic liquid and the identification information of the core of the atomizer. The identification information of the aromatic liquid may include the composition of the aromatic liquid and the quantity of aromatic liquid. The identification information of the core of the atomizer may include the material of the heating wires, the resistance values of the heating wires, and the information regarding the aromatic liquid suitable for the core of the atomizer. The electronic cigarette controller may include the central electronic cigarette controller 5, which may control the display of this information on a display device (not shown in the figures) using the electric power supply device of the electronic cigarette. During atomizer use, the recording chip 1 may preferably log the operating information of the atomizer, and send such information on number of uses and service life to the electronic cigarette controller 5. When the number of uses and/or the service life exceed the predefined threshold values, the electronic cigarette controller 5 may cease supplying electric power to the atomizer and/or propose the replacement of the core of the atomizer to the user.

The threshold values of the number of uses and of the service life may be selected or adjusted based on need and/or component life. In some embodiments, the threshold values may be defined, for example at 300 uses and 1000 hours. The proximity of the end of the abovementioned service life can be established when the residual values of the number of uses and/or of the service life represent 10% or 5% of the total values defined.

In some embodiments, recording chip 1 may be designed and selected depending on need by a person skilled in the art. Such person skilled in the art may design the physical shape and the placement of its installation depending on his need, and may connect it with the electronic cigarette controller in a wired or wireless configuration.

An electronic cigarette atomizer may be equipped with a recording chip 1; an electronic cigarette may be equipped with such atomizer embodiments. Recording chip 1 may be configured to acquire the identification information and the operating information; to identify and authenticate the identification information of the aromatic liquid and the identification information of the atomizer core; to check the aging of the core of the atomizer based on its service life and the number of uses of the atomizer core; to prevent the occurrence of atomizer leaks, which may be due to a poor match of the atomizer core or aging problems of the atomizer core; and/or to generally improve the safety of the atomizer of the electronic cigarette.

An ordinary electronic cigarette atomizer may typically include an atomizer upper cover, an atomizer baseplate, and an atomizer outer cylinder. Disposable electronic cigarette atomizers may further comprise an aromatic liquid cartridge. Said atomizer upper cover and said atomizer outer cylinder may be connected to the top of the atomizer outer cylinder. Said atomizer baseplate and said atomizer outer cylinder may be connected to the lower portion of the upper atomizer cylinder. The aromatic liquid cartridges may be generally arranged in said external atomizer cylinder. A portion of said recording chip may be arranged on the aromatic liquid cartridge. The lower portion of said atomizer baseplate may be connected to the electric power supply device of the electronic cigarette. The electric power supply device of the electronic cigarette may be equipped with an electronic cigarette controller, and said recording chip may be connected to the electronic cigarette controller.

In preferred embodiments, the identification of the aromatic liquid cartridge may be facilitated by the arrangement of a portion of the electronic chip on the aromatic liquid cartridge. In this manner, the aromatic liquid cartridge may be matched to the electronic cigarette atomizer for operation. This may prevent damage to the electronic cigarette due to incorrect aromatic liquid cartridge use.

Recording chip 1 may include a first information recorder 2, configured to acquire and record the identification information of the atomizer; a second information recorder 3 configured to acquire and record the operating information of the atomizer, which may include the service life and the number of uses of the core of the atomizer; an information transmitter 4, configured to send to the electronic cigarette controller the acquired identification information recorded operating information. Said first information recorder 2 may utilize an information marker and an information identifier, and said information marker may be connected to said aromatic liquid cartridge. When an aromatic liquid cartridge is installed with said electronic cigarette atomizer, said information identifier arranged on the electronic cigarette atomizer may identify said information marker installed on the aromatic liquid cartridge. In this manner, the first information recorder 2 may acquire the identification information of the aromatic liquid cartridge and, depending on said identification information of the aromatic liquid cartridge, it may confirm and record such identification information.

In a preferred embodiment, the dimensions of recording chip 1 may be relatively small so that it can be easily disposed in an atomizer and practically connected to the aromatic liquid cartridge. The connection to the atomizer may be facilitated via a connection of the electronic cigarette controller through the wiring of the three-pole electric power supply.

In one embodiment, the outer wall or the lower portion of the aromatic liquid cartridge may be equipped with a barcode, and the inner wall of the outer cylinder of the atomizer or the lower portion of the atomizer may be equipped with a corresponding barcode identifier. When the cartridge is installed in the atomizer, the barcode identifier may identify the barcode and acquire the identification information of the corresponding atomizer. A second information recorder 3 may read and acquire the operating information of the atomizer. The information transmitter 4 may send the acquired identification information and the recorded operating information to the electronic cigarette controller 5.

In another embodiment, the outer wall or the lower portion of the aromatic liquid cartridge may be equipped with a QR code, and the inner wall of the outer cylinder of the atomizer or the lower portion of the atomizer may be equipped with a corresponding QR code identifier. When the cartridge is installed in the atomizer, the QR code identifier may identify the QR code and may acquire the corresponding identification information of the atomizer. A second information recorder 3 may read and acquire the operating information of the atomizer. The information transmitter 4 may send the acquired identification information and the recorded operating information to the electronic cigarette controller 5.

The recording of information by barcode and QR code may permit the space occupied by the information marker to be reduced and may facilitate the reading of the information.

In another embodiment, the outer wall or the lower portion of the aromatic liquid cartridge may be equipped with a magnetic stripe, and the inner wall of the outer cylinder of the atomizer or the lower portion of the atomizer may be equipped with the corresponding magnetic stripe identifier. When the cartridge is installed in the atomizer, the magnetic stripe identifier may identify the magnetic stripe and may acquire the corresponding identification information of the atomizer. A second information recorder 3 may read and acquire the operating information of the atomizer. The information transmitter 4 may send the acquired identification information and the recorded operating information to the electronic cigarette controller 5.

The use of the magnetic stripe mode for recording the information may improve the safety and reading effectiveness of the information to be increased.

Preferably, said second information recorder 3 may be connected to said recording chip 1.

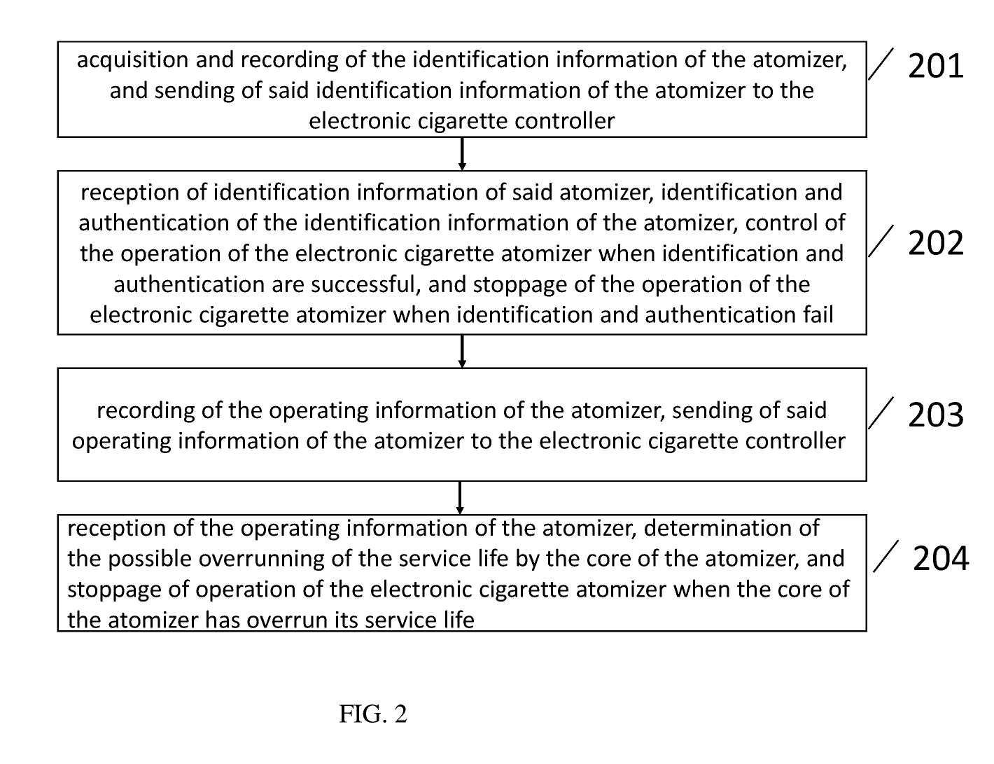

Referring to FIG. 2, a method for controlling the electronic cigarette equipped with said electronic cigarette atomizer may include:

a step 201: acquiring and recording of the identification information of the atomizer, and sending said identification information of the atomizer to the electronic cigarette controller;

a step 202: receiving of identification information of said atomizer, identification and authentication of the identification information of the atomizer, including at least the identification information of aromatic liquid and the identification information of the core of the atomizer; controlling of the operation of the electronic cigarette atomizer when identification and authentication are successful; and stopping of the operation of the electronic cigarette atomizer when identification and authentication fail;

a step 203: recording the operating information of the atomizer; sending of said operating information of the atomizer, including at least the service life and the number of uses of the core of the atomizer, to the electronic cigarette controller;

a step 204: receiving the operating information of the atomizer; determining of a possible service life overrun by the core of the atomizer; and stopping operation of the electronic cigarette atomizer if the core of the atomizer has overrun its service life.

Preferably, when the identification information of said electronic cigarette atomizer is received, the identification information of the atomizer is compared with the predefined identification information of the electronic cigarette atomizer to assess a proper match between the atomizer core and the aromatic liquid. If the match is proper, the operation of the electronic cigarette atomizer may be controlled based on the identification information of the electronic cigarette atomizer received by, for example, adjusting of the operating power, and adjusting the current and the temperature of the atomizer core.

Preferably, after operating information of the electronic cigarette atomizer is received, it is compared with corresponding predefined threshold values of duration and threshold values of number of uses. If the operating information of the atomizer approaches or exceeds the predefined threshold values of duration and/or threshold values of number of uses, operation of the electronic cigarette atomizer may be stopped; an indication suggesting replacement of the core of the atomizer may be displayed and/or otherwise provided prior to such stoppage.

Methods for controlling a electronic cigarette atomizer equipped with a recording chip are substantially similar to disclosed methods for controlling an electronic cigarette with electronic cigarette atomizer equipped with the abovementioned recording chip.

Although the foregoing embodiments have been described in detail by way of illustration and example for purposes of clarity of understanding, it will be readily apparent to those of ordinary skill in the art in light of the description herein that certain changes and modifications may be made thereto without departing from the spirit or scope of the appended claims. It is also to be understood that the terminology used herein is for the purpose of describing particular aspects only, and is not intended to be limiting, since the scope of the present invention will be limited only by the appended claims.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only,” and the like in connection with the recitation of claim elements, or use of a “negative” limitation. As will be apparent to those of ordinary skill in the art upon reading this disclosure, each of the individual aspects described and illustrated herein has discrete components and features which may be readily separated from or combined with the features of any of the other several aspects without departing from the scope or spirit of the disclosure. Any recited method can be carried out in the order of events recited or in any other order that is logically possible. Accordingly, the preceding merely provides illustrative examples. It will be appreciated that those of ordinary skill in the art will be able to devise various arrangements which, although not explicitly described or shown herein, embody the principles of the disclosure and are included within its spirit and scope.

Furthermore, all examples and conditional language recited herein are principally intended to aid the reader in understanding the principles of the invention and the concepts contributed by the inventors to furthering the art, and are to be construed without limitation to such specifically recited examples and conditions. Moreover, all statements herein reciting principles and aspects of the invention, as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary configurations shown and described herein.

In this specification, various preferred embodiments have been described with reference to the accompanying drawings. It will be apparent, however, that various other modifications and changes may be made thereto and additional embodiments may be implemented without departing from the broader scope of the claims that follow. The specification and drawings are accordingly to be regarded in an illustrative rather than restrictive sense. The foregoing constitutes exemplary embodiment of the present disclosure. It shall be understood that a person skilled in the art may, provided he does not depart from said principles of the present disclosure, carry out improvements and adjustments, these improvements and adjustments are considered to be covered by the scope of protection of the present disclosure

We claim:

1. An electronic cigarette atomizer comprising a recording chip, said recording chip configured to:
record identification information of the electronic cigarette atomizer, said identification information of the electronic cigarette atomizer comprising identification information of an aromatic liquid and identification information of a core of the atomizer;
send said identification information of the electronic cigarette atomizer to an electronic cigarette controller;
record operating information of the electronic cigarette atomizer, said operating information comprising service life and number of uses of the core of the atomizer; and
send said operating information to the electronic cigarette controller.

2. The electronic cigarette atomizer of claim 15, wherein the identification information of the core of the atomizer comprises:
information concerning a material of heating wires of the core of the atomizer;
information concerning resistance values of the heating wires of the core of the atomizer; and
information concerning aromatic liquids suitable for the core of the atomizer.

3. The electronic cigarette atomizer of claim 2, wherein the recording chip comprises:
a first information recorder configured to record the identification information of the electronic cigarette atomizer;
a second information recorder configured to record the operating information; and
an information transmitter configured to send the recorded identification information of the electronic cigarette atomizer and the operating information to the electronic cigarette controller.

4. The electronic cigarette atomizer of claim 3, further comprising an aromatic liquid cartridge, wherein said aromatic liquid cartridge equipped with the first information recorder.

5. The electronic cigarette atomizer of claim 3, further comprising an information identifier, wherein:
an information marker is provided with an aromatic liquid cartridge; and
when the aromatic liquid cartridge with the information marker is installed with said electronic cigarette atomizer, said information identifier is configured to:
identify said information marker; and
acquire the identification information of the aromatic liquid in the aromatic liquid cartridge from the information marker.

6. The electronic cigarette atomizer of claim 5, wherein:
said information marker is a barcode, a QR code or a magnetic stripe; and
said information identifier is a barcode identifier, a QR code identifier, or a magnetic stripe identifier.

7. The electronic cigarette atomizer of claim 3, wherein said second information recorder is configured to be connected to said core of the atomizer.

8. The electronic cigarette atomizer of claim 2, wherein said identification information of the aromatic liquid comprises information on the composition and the quantity of the aromatic liquid.

9. An electronic cigarette comprising an electronic cigarette atomizer equipped with a recording chip, and an electronic cigarette controller, wherein said recording chip is configured to:
record identification information of the electronic cigarette atomizer, said identification information comprising information on an aromatic liquid and information on a core of the atomizer;
send said identification information to the electronic cigarette controller;
record operating information of the electronic cigarette atomizer, said operating information comprising service life and number of uses of the core of the atomizer; and
send said operating information on the atomizer to the electronic cigarette controller.

10. The electronic cigarette of claim 9, wherein the information on the core of the atomizer comprises:
information concerning a material of heating wires of the core of the atomizer;
information concerning resistance values of the heating wires of the core of the atomizer; and
information concerning aromatic liquids suitable for the core of the atomizer.

11. The electronic cigarette of claim 10, wherein said electronic cigarette controller is configured to:
receive the identification information of the electronic cigarette atomizer from the recording chip;
identify and authenticate the received identification information;
control the operation of the electronic cigarette atomizer if the identification and authentication are successful;
prevent the operation of the electronic cigarette atomizer if the identification and authentication fail;
receive the operating information of the atomizer from the recording chip;
determine if the core of the atomizer is approaching or has exceeded its service life; and
stop the operation of the electronic cigarette atomizer if the core of the atomizer has exceeded its service life.

12. The electronic cigarette of claim 11, wherein the electronic cigarette controller further comprises an identifier-authenticator configured to:
receive the identification information;
compare the identification information with predefined identification information of the electronic cigarette atomizer to assess a proper match between the atomizer core and the aromatic liquid; and
if the match is assessed be proper, regulate operating parameters of the core of the atomizer depending on the received identification information to control the operation of the electronic cigarette atomizer,
wherein said operating parameters comprise:
an operating power;
a current of the core of the atomizer; and
a temperature of the core of the atomizer.

13. The electronic cigarette of claim 11, wherein the electronic cigarette controller further comprises a service life overrunning controller configured to:
acquire the operating information;
compare the operating information with predefined threshold values of duration and/or threshold values of number of uses; and
interrupt the operation of the electronic cigarette atomizer if the operating information exceeds the predefined threshold values of duration and/or threshold values of number of uses.

14. The electronic cigarette of claim 20, further comprising a user-perceptible output indicator, wherein the electronic cigarette controller is further configured to control the user-perceptible output indicator to:
provide an indication of the results of the identification and authentication if the identification information of the atomizer is identified and authenticated; and/or
provide an indication that the core of the atomizer is approaching or has exceeded its service life if the operating information exceeds the predefined threshold values of duration and/or threshold values of number of uses.

15. The electronic cigarette of claim 14, wherein the user-perceptible output indicator comprises at least one of:
a display screen;
an indicator light;
an auditory output element; and
a tactile feedback element.

16. A method for controlling an atomizer of electronic cigarette, the method comprising:
acquiring and recording identification information of the electronic cigarette atomizer, said identification information of the electronic cigarette atomizer comprising identification information of an aromatic liquid and identification information of a core of the atomizer;
sending said identification information of the electronic cigarette atomizer to an electronic cigarette controller;
acquiring and recording operating information of the electronic cigarette atomizer, said operating information comprising service life and number of uses of the core of the atomizer; and
sending said operating information to the electronic cigarette controller.

17. The method of claim 16, further comprising:
receiving the identification information of the electronic cigarette atomizer;
identifying and authenticating the received identification information of the electronic cigarette atomizer;
controlling the operation of the electronic cigarette atomizer if the identification and authentication are successful;
preventing the operation of the electronic cigarette atomizer if the identification and authentication fail;
receiving the operating information of the atomizer;
determining if the core of the atomizer is approaching or has exceeded its service life; and
stopping the operation of the electronic cigarette atomizer if the core of the atomizer has exceeded its service life.

18. The method of claim 17, wherein the identification information of the core of the atomizer comprises:
information concerning a material of heating wires of the core of the atomizer;
information concerning resistance values of the heating wires of the core of the atomizer; and
information concerning aromatic liquids suitable for the core of the atomizer.

19. The method of claim 18, further comprising:
comparing the identification information of the electronic cigarette atomizer with predefined identification information of the electronic cigarette atomizer to assess a proper match between the atomizer core and the aromatic liquid; and
if the match is assessed be proper, regulating operating parameters of the core of the atomizer depending on the received identification information to control the operation of the electronic cigarette atomizer,
wherein said operating parameters comprise:
an operating power;
a current of the core of the atomizer; and
a temperature of the core of the atomizer.

20. The method of claim 18, further comprising:
comparing the operating information with predefined threshold values of duration and/or threshold values of number of uses; and
interrupting the operation of the electronic cigarette atomizer and/or providing and indication to the user if the operating information exceeds the predefined threshold values of duration and/or threshold values of number of uses.
